(19) United States
(12) Patent Application Publication
(10) Pub. No.: US 2007/0142431 A1
(43) Pub. Date: Jun. 21, 2007

(54) Quinoline 4-carboxamide derivatives and their use as neurokinin 3 (nk-3) receptor antagonists

(75) Inventors: Wai Ngor Chan, Essex (GB); Paul William Smith, Essex (GB); Paul Adrian Wyman, Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: SmithKline Beecham Corporation

(21) Appl. No.: 10/567,655

(22) PCT Filed: Aug. 5, 2004
(86) PCT No.: PCT/EP04/08842

§ 371 Date: Jul. 18, 2006

(30) Foreign Application Priority Data
Aug. 8, 2003 (GB) 0318727.5

(51) Int. Cl. A61K 31/4709; C07D 403/02

(52) U.S. Cl. 514/314; 546/169

(57) ABSTRACT

The invention relates to novel quinoline derivatives, processes for their preparation, pharmaceutical compositions containing them and their use as medicaments particularly in treating disorders of the central nervous system (CNS).

The present invention relates to novel quinoline derivatives, processes for their preparation, pharmaceutical compositions containing them and their use as medicaments particularly in treating disorders of the central nervous system (CNS).

The mammalian peptide Neurokinin B (NKB) belongs to the Tachykinin (TK) peptide family which also include Substance P (SP) and Neurokinin A (NKA). Pharmacological and molecular biological evidence has shown the existence of three subtypes of TK receptor (NK₁, NK₂ and NK₃). NKB binds preferentially to the NK₃ receptor although it also recognises the other two receptors with lower affinity (Maggi et al, 1993, J. Auton. Pharmacol., 13, 23-93).

Selective peptidic NK₃ receptor antagonists are known (Drapeau, 1990 Regul. Pept, 31, 125-135) and findings with peptidic NK₃ receptor agonists suggest that NKB, by activating the NK₃ receptor, has a key role in the modulation of neural input in airways, skin, spinal cord and nigro-striatal pathways (Myers and Undem, 1993, J.Physiol., 470, 665-679; Counture et al., 1993, Regul. Peptides, 46, 426-429; McCarson and Krause, 1994, J. Neurosci., 14 (2), 712-720; Arenas et al. 1991, J.Neurosci., 11, 2332-8).

WO 97/19926 discloses quinoline derivatives with activity as NK₃ receptor antagonists.

We have now identified a group of quinoline derivatives that exhibit advantageous properties.

Therefore according to a first aspect, the invention provides a compound of formula (I), a pharmaceutically acceptable salt, solvate or prodrug thereof

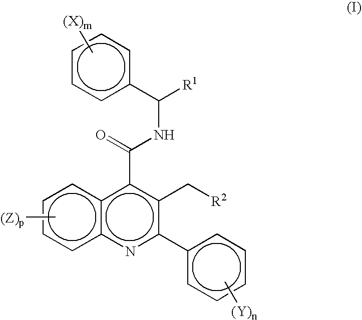

wherein

R¹ is C₁₋₆alkyl (preferably ethyl), C₃₋₆cycloalkyl (preferably cyclopropyl) or acetyl;

R² is N-linked pyrazolyl, triazolyl or tetrazolyl each of which may be substituted by

C₁₋₄alkyl or perfluoro C₁₋₄alkyl;

m, n and p, which may be the same or different, are 0, 1 or 2; and

X, Y and Z are fluoro.

Preferably R¹ is cyclopropyl.

Preferably R² is N-linked triazolyl or N-linked tetrazolyl. More preferably R² is N-2 linked triazolyl or N-2 linked tetrazolyl. Preferably R² is unsubstituted N-2 linked triazolyl or N-2 linked tetrazolyl

Preferably p is 0.

Preferably m and n, which may be the same or different, are 0 or 1. More preferably either a) m is 0 and n is 1,or b) m is 1 and n is 0.

When m and/or n are 1, preferably X and/or Y are attached to the meta-position of the phenyl groups.

Preferably the compound according to the first aspect is of formula (Ia):

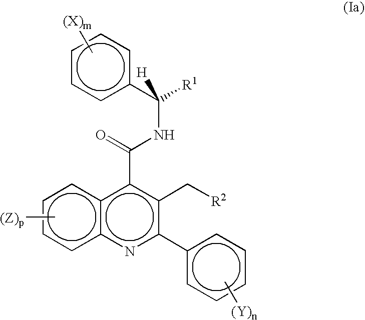

It will be appreciated that the present invention is intended to include compounds having any combination of the preferred groups listed hereinbefore.

Preferably the invention provides a compound of formula (I) or (Ia) wherein

R¹ is C₁₋₆alkyl (preferably ethyl) or C₃₋₆cycloalkyl (preferably cyclopropyl);

R² is N-linked triazolyl or N-linked tetrazolyl each of which may be substituted by

C₁₋₄alkyl or perfluoro C₁₋₄alkyl;

m and n, which may be the same or different, are 0, 1 or 2;

p is 0; and

X and Y are fluoro.

More preferably the invention provides a compound of formula (I) or (Ia) wherein

R¹ is ethyl or cyclopropyl;

R² is N-2 linked triazolyl or N-2 linked tetrazolyl;

m and n, which may be the same or different, are 0 or 1;

p is 0; and

X and Y are fluoro.

Still more preferably the invention provides a compound of formula (I) or (Ia) wherein

R¹ is cyclopropyl;

R² is unsubstituted N-2 linked triazolyl or N-2 linked tetrazolyl;

either a) m is 0 and n is 1, or b) m is 1 and n is 0;

p is 0; and

X and Y are fluoro attached to the meta-position of the phenyl groups.

Preferred compounds of formula (I) are:

2-(3-Fluoro-phenyl)-3-[1,2,3]triazol-2-ylmethyl-quinoline-4-carboxylic acid ((S)-1-cyclopropyl-1-phenyl-methyl)-amide (Example 1);

2-Phenyl-3-[1,2,3]triazol-2-ylmethyl-quinoline-4-carboxylic acid [(S)-1-cyclopropyl-1-(3-fluoro-phenyl)-methyl]-amide (Example 17);

2-Phenyl-3-tetrazol-2-ylmethyl-quinoline-4-carboxylic acid [(S)-1-cyclopropyl-1-(3-fluoro-phenyl)-methyl]-amide (Example 35); and

2-(3-Fluoro-phenyl)-3-tetrazol-2-ylmethyl-quinoline-4-carboxylic acid ((S)-1-cyclopropyl-1-phenyl-methyl)-amide (Example 45).

Suitable pharmaceutically acceptable salts of the compounds of formula (I) include basic salts with the appropriate acid for example organic carboxylic acids such as acetic, lactic, tartaric, malic, isethionic, lactobionic and succinic acids; organic sulfonic acids such as methanesulfonic, ethanesulfonic, benzenesulfonic and p-toluenesulfonic acids and inorganic acids such as hydrochloric, sulfuric, phosphoric and sulfonic acids and the like. Some of the compounds of this invention may be crystallised or recrystallised from solvents such as aqueous and organic solvents. In such cases solvates may be formed. This invention includes within its scope stoichiometric solvates including hydrates as well as compounds -containing variable amounts of water that may be produced by processes such as lyophilisation.

The pharmaceutically acceptable solvates of the compounds of formula (I) include the hydrates thereof.

Hereinafter, compounds, their pharmaceutically acceptable salts and their solvates defined in the first aspect of the invention are referred to as “compounds of the invention”.

The compounds of the invention include polymorphs thereof.

The compounds of the invention may exist in one or more tautomeric forms. All tautomers and mixtures thereof are included in the scope of the present invention.

Compounds of the invention may exist in the form of optical isomers, e.g. diastereoisomers and mixtures of isomers in all ratios, e.g. racemic mixtures. The invention includes all such forms, in particular the pure isomeric forms. The different isomeric forms may be separated or resolved one from the other by conventional methods, or any given isomer may be obtained by conventional synthetic methods or by stereospecific or asymmetric syntheses.

Since the compounds of the invention are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and preferably from 10 to 59% of a compound of the invention.

The compounds of the invention are potent and selective NK₃ antagonists. In addition the compounds of the invention show pharmaceutically advantageous properties over the compounds disclosed in WO 97/19926. In particular, the compounds of the invention show increased in vivo brain exposure than the compounds disclosed in WO 97/19926. It will be appreciated that increased brain exposure is an important property in compounds for treating disorders of the CNS.

Compounds of the invention may be prepared, in known manner in a variety of ways. In the following reaction schemes and hereafter, unless otherwise stated R¹, R², X, Y, Z, m, n and p are as defined in the first aspect. These processes form further aspects of the invention.

Throughout the specification, general formulae are designated by Roman numerals (I), (II), (III), (IV) etc. Subsets of these general formulae are defined as (Ia), (Ib), (Ic) etc. . . . (IVa), (IVb), (IVc) etc.

Compounds of formula (I) may be prepared according to reaction scheme 1 from compounds of formula (II) by reaction with compounds of formula (III) using amide coupling reagents such as HATU. Preferably the reaction is carried out in the presence of a suitable base such as diisopropylethylamine in a suitable solvent such as DMF.

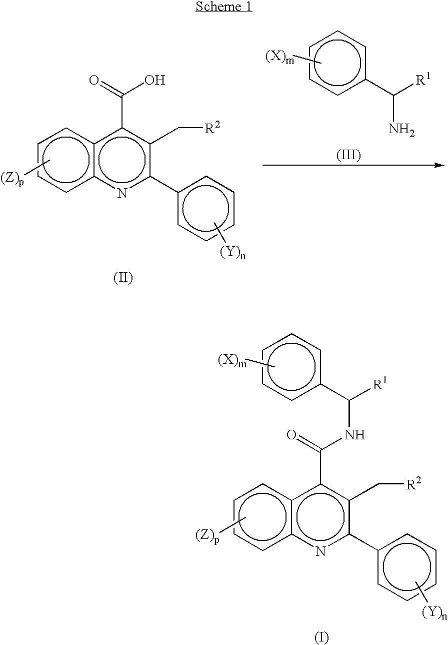

Compounds of formula (II) may be prepared according to reaction scheme 2 from compounds of formula (IV) by base catalysed hydrolysis. A suitable base for this transformation is lithium hydroxide.

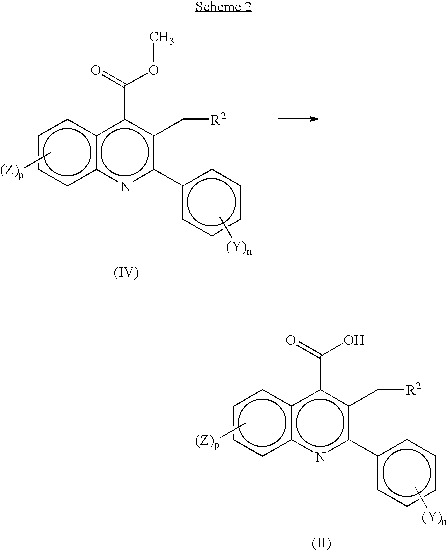

Compounds of formula (IV) may be prepared according to reaction scheme 3 from compounds of formula (V) by reaction with a nitrogen containing heterocycle such as 1,2,3 triazole. Preferably the reaction is carried out in the presence of a base such as sodium hydride in a suitable solvent such as dimethylformamide.

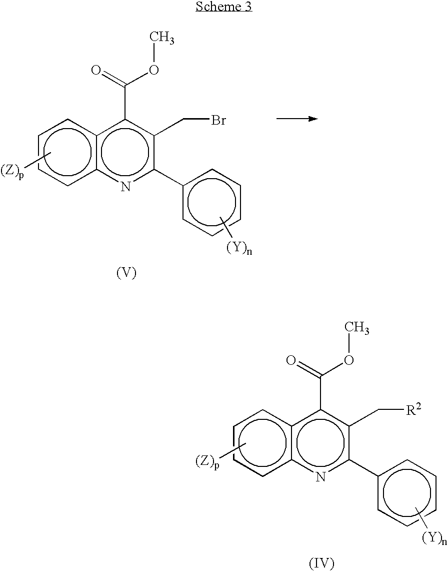

Compounds of formula (V) may be prepared according to reaction scheme 4 from compounds of formula (VI) using a brominating reagent. A suitable brominating reagent is N-bromo succinimide, a suitable solvent is carbon tetrachloride.

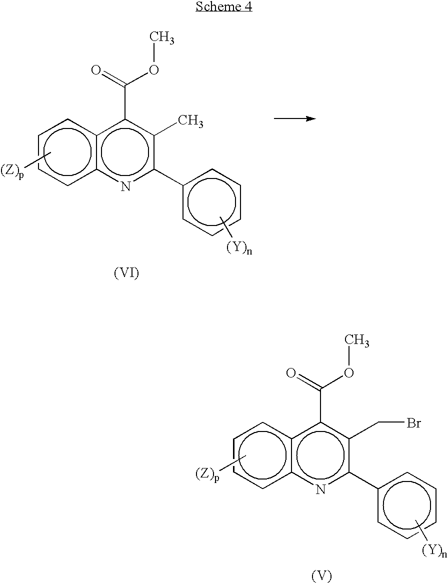

Compounds of formula (VI) may be prepared according to reaction scheme 5 by esterification of compounds of formula (VII). An intermediate carboxylic acid chloride may be prepared using suitable reagents such as oxalyl chloride, which can then be converted to the carboxylic acid ester by reaction with methanol.

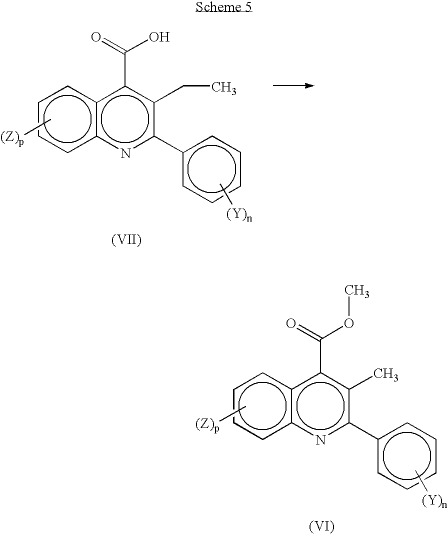

Compounds of formula (VII) may be prepared according to reaction scheme 6 by reacting compounds of formula (VIII) with compounds of formula (IX). Preferably the reaction is carried out in the presence of a suitable acid such as concentrated hydrochloric acid in a suitable solvents such as glacial acetic acid at an elevated temperature, suitably 105 deg C.

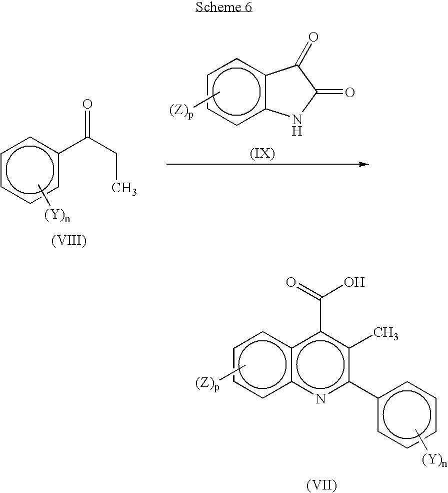

Compounds of formula (III) (see scheme 1) may be prepared according to reaction scheme 7 by reducing compounds of formula (X) using conditions such as hydrogenation and a supported palladium catalyst.

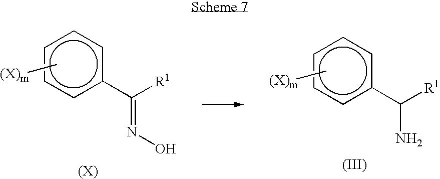

Compounds of formula (X) may be prepared according to reaction scheme 8 by reacting compounds of formula (XI) with hydroxylamine in the presence of a base. A suitable base for this transformation is potassium hydroxide.

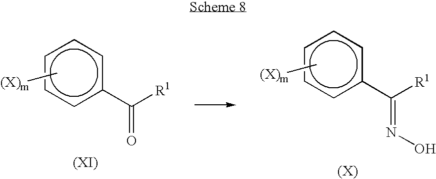

Compounds of formula (IIIa), i.e. compounds of formula (III) where R¹ is (S)-cyclopropyl, may be prepared according to reaction scheme 9 from compounds of formula (XII) by reaction with periodic acid in the presence of a suitable base such as methylamine.

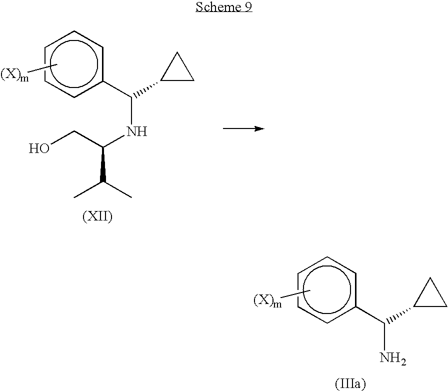

Compounds of formula (XII) may be prepared according to reaction scheme 10 from compounds of formula (XIII) by reaction with cyclopropyl lithium (generated in situ from cyclopropyl bromide and tert butyl lithium).

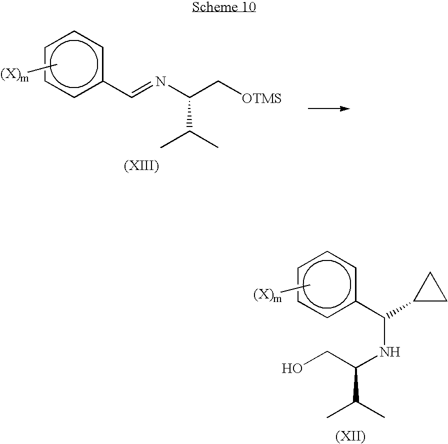

Compounds of formula (XIII) may be prepared according to reaction scheme 11 from commercially available benzaldehydes (XIV) by reaction with valinol followed by protection of the alcohol functionality as its trimethylsilyl ether.

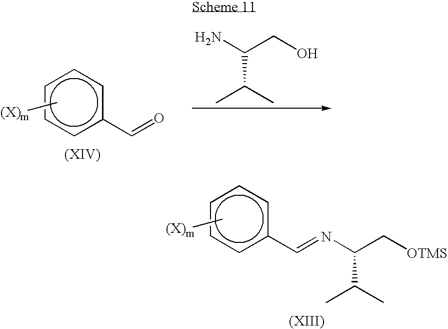

Further details for the preparation of compounds of formula (I) are found in the examples section hereinafter.

The compounds of the invention may be prepared singly or as compound libraries comprising at least 2, for example 5 to 1,000 compounds, and more preferably 10 to 100 compounds. Libraries of compounds of the invention may be prepared by a combinatorial ‘split and mix’ approach or by multiple parallel synthesis using either solution phase or solid phase chemistry, by procedures known to those skilled in the art. Thus according to a further aspect there is provided a compound library comprising at least 2 compounds of the invention.

As discussed hereinabove findings with peptidic NK₃ receptor agonists suggest that NKB, by activating the NK₃ receptor, has a key role in the modulation of neural input in airways, skin, spinal cord and nigro-striatal pathways.

Therefore, according to a further aspect, the invention provides a compound of the invention for use as a medicament, preferably a human medicament.

According to a still further aspect the invention provides the use of a compound of the invention in the manufacture of a medicament for treating or preventing a disease or condition mediated by modulation of the NK₃ receptor.

According to a further aspect, the invention provides a method for treating or preventing a disease or condition mediated by modulation of the NK3 receptor in mammals (preferably humans), which comprises administration to the mammal in need of such treatment, an effective amount of a compound of the invention.

Preferred diseases or conditions mediated by modulation of the NK₃ receptor are CNS disorders such as depression (which term includes bipolar (manic) depression (including type I and type II), unipolar depression, single or recurrent major depressive episodes with or without psychotic features, catatonic features, melancholic features, atypical features (e.g. lethargy, over-eating/obesity, hypersomnia) or postpartum onset, seasonal affective disorder and dysthymia, depression-related anxiety, psychotic depression, and depressive disorders resulting from a general medical condition including, but not limited to, myocardial infarction, diabetes, miscarriage or abortion); anxiety disorders (including generalised anxiety disorder (GAD), social anxiety disorder (SAD), agitation, tension, social or emotional withdrawal in psychotic patients, panic disorder, and obsessive compulsive disorder); phobias (including agoraphobia and social phobia); psychosis and psychotic disorders (including schizophrenia, schizo-affective disorder, schizophreniform diseases, acute psychosis, alcohol psychosis, autism, delerium, mania (including acute mania), manic depressive psychosis, hallucination, endogenous psychosis, organic psychosyndrome, paranoid and delusional disorders, puerperal psychosis, and psychosis associated with neurodegenerative diseases such as Alzheimer's disease); post-traumatic stress disorder; attention deficit hyperactive disorder (ADHD); cognitive impairment (e.g. the treatment of impairment of cognitive functions including attention, orientation, memory (memory disorders, amnesia, amnesic disorders and age-associated memory impairment) and language function, and including cognitive impairment as a result of stroke, Alzheimer's disease, Aids-related dementia or other dementia states, as well as other acute or sub-acute conditions that may cause cognitive decline such as delirium or depression (pseudodementia states)); convulsive disorders such as epilepsy (which includes simple partial seizures, complex partial seizures, secondary generalised seizures, generalised seizures including absence seizures, myoclonic seizures, clonic seizures, tonic seizures, tonic clonic seizures and atonic seizures); psychosexual dysfunction (including inhibited sexual desire (low libido), inhibited sexual arousal or excitement, orgasm dysfunction, inhibited female orgasm and inhibited male orgasm, hypoactive sexual desire disorder (HSDD), female sexual desire disorder (FSDD), and sexual dysfunction side-effects induced by treatment with antidepressants of the SSRI-class); sleep disorders (including disturbances of circadian rhythm, dyssomnia, insomnia, sleep apnea and narcolepsy); disorders of eating behaviours (including anorexia nervosa and bulimia nervosa); neurodegenerative diseases (such as Alzheimer's disease, ALS, motor neurone disease and other motor disorders such as Parkinson's disease (including relief from locomotor deficits and/or motor disability, including slowly increasing disability in purposeful movement, tremors, bradykinesia, hyperkinesia (moderate and severe), akinesia, rigidity, disturbance of balance and co-ordination, and a disturbance of posture), dementia in Parkinson's disease, dementia in Huntington's disease, neuroleptic-induced Parkinsonism and tardive dyskinesias, neurodegeneration following stroke, cardiac arrest, pulmonary bypass, traumatic brain injury, spinal cord injury or the like, and demyelinating diseases such as multiple sclerosis and amyotrophic lateral sclerosis); withdrawal from abuse of drugs including smoking cessation or reduction in level or frequency of such activities (such as abuse of cocaine, ethanol, nicotine, benzodiazepines, alcohol, caffeine, phencyclidine and phencyclidine-like compounds, opiates such as cannabis, heroin, morphine, sedative, hypnotic, amphetamine or amphetamine-related drugs such as dextroamphetamine, methylamphetamine or a combination thereof); pain (which includes neuropathic pain (including diabetic neuropathy; sciatica; non-specific lower back pain; multiple sclerosis pain; pain associated with fibromyalgia or cancer; AIDS-related and HIV-related neuropathy; chemotherapy-induced neuropathy; neuralgia, such as post-herpetic neuralgia and trigeminal neuralgia; sympathetically maintained pain and pain resulting from physical trauma, amputation, cancer, toxins or chronic inflammatory conditions such as rheumatoid arthritis and osteoarthritis; reflex sympathetic dystrophy such as shoulder/hand syndrome), acute pain (e.g. musculoskeletal pain, post operative pain and surgical pain), inflammatory pain and chronic pain, pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia), pain associated with migrane, and non-cardiac chest pain); and certain CNS-mediated disorders (such as emesis, irritable bowel syndrome and non-ulcer dyspepsia).

More preferred diseases or conditions mediated by modulation of the NK3 receptor are depression; anxiety disorders; phobias; psychosis and psychotic disorders; post-traumatic stress disorder; attention deficit hyperactive disorder (ADHD); withdrawal from abuse of drugs including smoking cessation or reduction in level or frequency of such activities; irritable bowel syndrome; cognitive impairment; convulsive disorders; psychosexual dysfunction; sleep disorders; disorders of eating behaviours; neurodegenerative diseases; pain; emesis; irritable bowel syndrome; and non-ulcer dyspepsia.

Still more preferred diseases or conditions mediated by modulation of the NK3 receptor depression; anxiety disorders; phobias; and psychosis and psychotic disorders (especially schizophrenia, schizo-affective disorder and schizophreniform diseases).

It will be appreciated that references herein to “treatment” extend to prophylaxis, prevention of recurrence and suppression or amelioration of symptoms (whether mild, moderate or severe) as well as the treatment of established conditions. The compound of the invention may be administered as the raw chemical but the active ingredient is preferably presented as a pharmaceutical formulation.

According to a further aspect, the invention provides a pharmaceutical composition comprising a compound of the invention, in association with one or more pharmaceutically acceptable carrier(s), diluents(s) and/or excipient(s). The carrier, diluent and/or excipient must be “acceptable” in the sense of being compatible with the other ingredients of the composition and not deleterious to the recipient thereof.

The compounds of the invention may be administered in conventional dosage forms prepared by combining a compound of the invention with standard pharmaceutical carriers or diluents according to conventional procedures well known in the art. These procedures may involve mixing, granulating and compressing or dissolving the ingredients as appropriate to the desired preparation.

The pharmaceutical compositions of the invention may be formulated for administration by any route, and include those in a form adapted for oral, topical or parenteral administration to mammals including humans.

The compositions may be formulated for administration by any route. The compositions may be in the form of tablets, capsules, powders, granules, lozenges, creams or liquid preparations, such as oral or sterile parenteral solutions or suspensions.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Suppositories will contain conventional suppository bases, e.g. cocoa-butter or other glyceride.

For parenteral administration, fluid unit dosage forms are prepared utilising the compound and a sterile vehicle, water being preferred. The compound, depending on the vehicle and concentration used, can be either suspended or dissolved in the vehicle. In preparing solutions the compound can be dissolved in water for injection and filter sterilised before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilised powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilisation cannot be accomplished by filtration. The compound can be sterilised by exposure to ethylene oxide before suspending in the sterile vehicle. Advantageously, a surfactant or wetting agent is included in the composition to facilitate uniform distribution of the compound.

The compositions may contain from 0.1% by weight, preferably from 10-60% by weight, of the active material, depending on the method of administration. Where the compositions comprise dosage units, each unit will preferably contain from 50-500 mg of the active ingredient. The dosage as employed for adult human treatment will preferably range from 10 to 3000 mg per day, for instance 1500 mg per day depending on the route and frequency of administration. Such a dosage corresponds to 0.1 to 50 mg/kg per day.

It will be recognised by one of skill in the art that the optimal quantity and spacing of individual dosages of a compound of the invention will be determined by the nature and extent of the condition being treated, the form, route and site of administration, and the particular mammal being treated, and that such optimums can be determined by conventional techniques. It will also be appreciated by one of skill in the art that the optimal course of treatment, i.e., the number of doses of a compound of the invention given per day for a defined number of days, can be ascertained by those skilled in the art using conventional course of treatment determination tests.

No toxicological effects are indicated when a compound of the invention is administered in the above-mentioned dosage range.

All publications, including, but not limited to, patents and patent applications cited in this specification, are herein incorporated by reference as if each individual publication were specifically and individually indicated to be incorporated by reference herein as though fully set forth.

The following non-limiting examples illustrate the present invention.

Abbreviations used
DBU—1,8-diazabicyclo[5.4.0]undec-7-ene
DMF—dimethylformamide
DIPEA—diisopropylethylamine
DMSO—dimethylsulphoxide
EDC—1-(3-dimethylaminopropyl) 3-ethylcarbodiimide hydrochloride
HATU—N-[(dimethylamino-1H-1,2,3-triazolo 4,5b pyridin-1-yl methylene] N-methyl methanaminium hexafluorophosphate N-oxide
HOBt—1-hydroxybenzotriazole hydrate
THF—tetrahydrofuran
TMS-CI—trimethylsilylchloride

¹H NMR spectra were recorded on a Bruker B-ACS 60 400 MHz, Bruker DPX 400 or a Bruker DPX 250. Chemical shifts are expressed in parts per million (ppm, δ units). Coupling constants (J) are in units of hertz (Hz). Splitting patterns describe apparent multiplicities and are designated as s (singlet), d (doublet), t (triplet), q (quartet), dd (double doublet), dt (double triplet), m (multiplet), br (broad).

Low-resolution mass spectra (MS) were recorded on a HP1100 series spectrometer; MS and liquid chromatography MS were recorded on a Micromass MS2 Platform LC spectrometer. All mass spectra were taken under electrospray ionisation (ESI), chemical ionisation (CI), electron impact (EI) or by fast atom bombardment (FAB) methods. All reactions were monitored by thin-layer chromatography on 0.25 mm E. Merck silica gel plates (60F-254), visualised with UV light, 5% ethanolic phosphomolybdic acid, p-anisaldehyde solution, aqueous potassium permanganate or potassium iodide/platinum chloride solution in water. Flash column chromatography was performed on silica gel.

Intermediates

### Intermediate 1: (S)-2-(Benzylidene-amino)-3-methyl-butan-1-ol

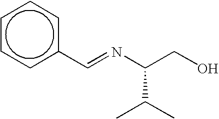

(S)-(+)-Valinol (4.16 g, 40.3 mmol, 1 eq) was dissolved in dichloromethane (60 mL), magnesium sulphate (20 g) added, the mixture cooled to 0° C. and treated dropwise with benzaldehyde (4.28 g, 40.3 mmol, 1 eq). Stirring was continued at 0° C. for 2 hrs and then at ambient temperature for 18 hrs. The reaction mixture was filtered and evaporated in vacuo to afford the title compound as a white solid (6.7 g, 87%); m/z (APCI): 192 [M+H]⁺.

### Intermediate 2: (S)-2-[(3-Fluoro-benzylidene)-amino]-3-methyl-butan-1-ol

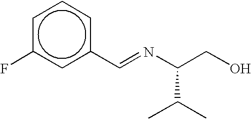

The title compound was prepared in a similar manner to intermediate 1 using 3-fluorobenzaldehyde.

### Intermediate 3: (S)-2-[(2-Fluoro-benzylidene)-aminol-3-methyl-butan-1-ol

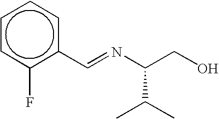

The title compound was prepared in a similar manner to intermediate 1 using 2-fluorobenzaldehyde.

### Intermediate 4: Benzylidene-((S)-2-methyl-1-trimethylsilanyl oxymethyl-propyl)-amine.

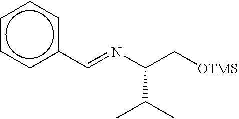

Intermediate 1 (6.7 g, 35 mmol, 1 eq) was dissolved in dry dichloromethane (60 mL) and treated with triethylamine (5.4 mL, 38.5 mmol, 1.1 eq) and TMS-CI (4.9 mL, 38.5 mmol, 1.1 eq) under argon. The mixture was stirred at ambient temperature for 72 hours, filtered and then evaporated to dryness. The residue was triturated with diethylether and the filtrate evaporated to dryness in vacuo to afford the title compound (8.43 g, 91%) as a colourless oil; ¹H NMR (400 MHz, CDCI₃) 0.01 (9H, s), 0.88-0.90 (6H, m), 1.87-1.95 (1H, m), 2.92-2.97 (1H, m), 3.59-3.64 (1H, m), 3.82-3.85 (1H, m), 7.22-7.37 (3H, m), 7.68-7.73 (2H, m), 8.17 (1H, s).

### Intermediate 5: (3-Fluoro-benzylidene)-((S)-2-methyl-1-trimethylsilanyloxymethyl-propyl)-amine

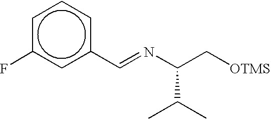

The title compound was prepared in a similar manner to intermediate 4 using intermediate 2 as starting material; ¹H NMR (400 MHz, CDCI₃) 0.01 (9H, s), 0.86-0.90 (6H, m), 1.87-1.95 (1H, m), 2.94-2.98 (1H, m), 3.58-3.63 (1H, m), 3.81-3.84 (1H, m), 7.04-7.06 (1H, m), 7.32-7.35 (1H, m), 7.42-7.48 (2H, m), 8.13 (1H, s).

### Intermediate 6: (2-Fluoro-benzylidene)-((S)-2-methyl-1-trimethylsilanyloxymethyl-propyl)-amine

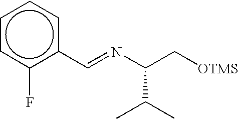

The title compound was prepared in a similar manner to intermediate 4 using intermediate 3 as starting material; ¹H NMR (400 MHz, CDCI₃) 0.01 (9H, s), 0.86-0.90 (6H, m), 1.87-1.95 (1H, m), 2.95-3.00 (1H, m), 3.58-3.62 (1H, m), 3.80-3.84 (1H, m), 6.99-7.04 (1H, t), 7.09-7.13 (1H, t), 7.31-7.33 (1H, m), 7.93-7.97 (1H, t), 8.45 (1H, s).

### Intermediate 7: (S)-2-[((S)-1-Cyclopropyl-1-phenyl-methyl)-amino]-3-methyl-butan-1-ol.

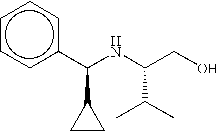

Cyclopropyl bromide (4.64 g, 38.4 mmol, 1.2 eq) was dissolved in dry diethylether (50 mL) under argon, cooled to −78° C. and treated with -tert-BuLi (45 mL of a 1.7M solution in pentane, 76.5 mmol, 2.4 eq). After 10 minutes, cooling was removed and the mixture stirred at room temperature for 1 hr. After recooling to −40° C., a solution of intermediate 4 (8.43 g, 32 mmol, 1 eq) in dry diethylether (40 mL) was added and stirring continued at −40° C. for 1.5 hrs. 5M HCl was added (50 mL) and the phases separated. The aqueous phase was washed with diethylether (discarded) and then basified with KOH pellets to pH>10 in the presence of diethylether. The organic phase was washed with water and brine and then evaporated to dryness in vacuo to afford the title compound as a colourless oil (6.42 g, 86%); ¹H NMR (400 MHz, CDCI₃) 0.13-0.15 (1H, m), 0.34-0.37 (2H, m), 0.60-0.70 (1H, m), 0.83 (3H, d, J=7 Hz), 0.91 (3H, d, J=7 Hz), 0.98-1.00 (1H, m), 1.71-1.77 (1H, m), 2.44-2.48 (1H, m), 3.00 (1H, d, J=8 Hz), 3.32 and 3.36 (1H, dd, J=5 and 11 Hz), 3.59 and 3.61 (1H, dd, J=5 and 11 Hz), 7.25-7.42 (5H, m); m/z(APCI): 234 [M+H]⁺.

### Intermediate 8: (S)-2-{[(S)-1-Cyclopropyl-1-(3-fluoro-phenyl)-methyl]-amino}-3-methyl-butan-1-ol

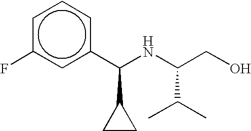

The title compound was prepared in a similar manner to intermediate 7 using intermediate 5 as starting material; ¹H NMR (400 MHz, CDCI₃) 0.15-0.17 (1H, m), 0.35-0.38 (2H, m), 0.65-0.67 (1H, m), 0.83 (3H, d, J=7 Hz), 0.91 (3H, d, J=7 Hz), 1.00-1.03 (1H, m), 1.70-1.77 (1H, m), 2.40-2.44 (1H, m), 2.99 (1H, d, J=9 Hz), 3.36 and 3.88 (1H, dd, J=5 and 11 Hz), 3.59 and 3.62 (1H, dd, J=5 and 11 Hz), 6.94-6.97 (1H, m), 7.03-7.08 (2H, m), 7.26-7.29 (1H, m).

### Intermediate 9: (S)-2-{[(S)-1-Cyclopropyl-1-(2-fluoro-phenyl)-methyl]-amino}-3-methyl-butan-1-ol

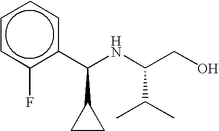

The title compound was prepared in a similar manner to intermediate 7 using intermediate 6 as starting material; ¹H NMR (400 MHz, CDCI₃) 0.18-0.19 (1H, m), 0.36-0.40 (2H, m), 0.66-0.68 (1H, m), 0.82 (3H, d, J=7 Hz), 0.89 (3H, d, J=7 Hz), 1.14-1.17 (1H, m), 1.69-1.75 (1H, m), 2.32-2.36 (1H, m), 3.27 (1H, d, J=9 Hz), 3.36 and 3.39 (1H, dd, J=5 and 11 Hz), 3.59 and 3.60 (1H, dd, J=5 and 11 Hz), 7.00-7.04 (1H, t), 7.12-7.14 (1H, t), 7.21-7.24 (1H, m), 7.35-7.37 (1H, t).

### Intermediate 10: (S)-1-Cyclopropyl-1-phenyl-methylamine (hydrochloride salt)

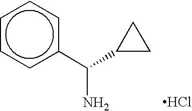

Intermediate 7 (1.67 g, 7.2 mmol,1 eq) was dissolved in methanol (20 mL) and aqueous methylamine (9mL of a 40% solution in water) added. This mixture was treated with a solution of H₅IO₆ (5.30 g, 23.3 mmol, 3.2 eq) in water (5 mL). An initial exotherm was observed (approx 50 deg C.). After 24 hrs at ambient temperature, some starting material was evident by tic (NH₃/MeOH/CH₂CI₂ 1:9:90), so the mixture was heated to reflux for 30 minutes. After cooling to room temperature a further portion of H₅IO₆ (1.8 g, 7.9 mmol, 1.1 eq) in water (5 mL) and aqueous methylamine (5 mL) were added and stirring continued for a further 18 hrs at ambient temperature. All insoluble material was removed by filtration and washed with methanol. The filtrate and washings were concentrated in vacuo and the residue partitioned between diethylether (×5) and water. The combined, organic extracts were concentrated to low volume in vacuo, treated with 5M HCl (10 mL) and stirred for 18 hrs at ambient temperature. After reduction to a small volume, the residue was washed with diethylether and then basified with KOH pellets (to pH>10) in the presence of diethylether. The phases were separated and the organic phase washed with water, saturated brine and dried over magnesium sulphate. The filtrate was treated with HCl (10 mL of a I M solution in ether) and the product collected by filtration (0.972 g, 74%); ¹H NMR [400 MHz, DMSO-d6] 0.36-0.38 (1H, m), 0.47-0.49 (1H, m), 0.60-0.65 (2H, m), 1.30-1.35 (1H, m), 3.54-3.58 (1H, m), 7.35-7.44 (3H, m), 7.55-7.58 (2H, m), 8.71 (3H, brs, exchangeable); [α]²⁸_{D}=+45.9° (c=1 in MeOH).

### Intermediate 11: (S)-1-Cyclopropyl-1-(3-fluoro-phenyl)-methylamine (hydrochloride salt)

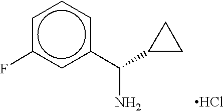

The title compound was prepared in a similar manner to intermediate 10 using intermediate 8 as starting material; ¹H NMR [400 MHz, DMSO-d6] 0.39-0.42 (1H, m), 0.47-0.51 (1H, m), 0.60-0.67 (2H, m), 1.29-1.35 (1H, m), 3.59-3.62 (1H, m), 7.20-7.24 (1H, m), 7.39-7.41 (1H, m), 7.45-7.51 (2H, m), 8.73 (3H, brs, exchangeable); [α]²⁵_{D}=+42.1° (c=1 in EtOH).

### Intermediate 12: (S)-1-Cyclopropyl-1-(2-fluoro-phenyl)-methylamine (hydrochloride salt)

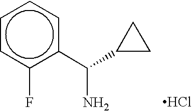

The title compound was prepared in a similar manner to intermediate 10 using intermediate 9 as starting material; ¹H NMR [400 MHz, DMSO-d6] 0.29-0.32 (1H, m), 0.49-0.52 (1H, m), 0.64-0.67 (2H, m), 1.37-1.43 (1H, m), 3.80-3.82 (1H, m), 7.24-7.32 (2H, m), 7.43-7.45 (1H, m), 7.78-7.80 (1H, m), 8.80 (3H, brs, exchangeable).

### Intermediate 13: (S)-3-Methyl-2-phenyl-butyric acid. (S)-1-phenylethylamine salt

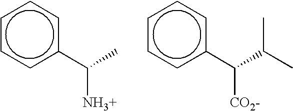

A solution of (S)-1-phenylethylamine (23.8 g, 200 mmol, 1 eq) and (RS)-isopropylphenylacetic acid (35 g, 200 mmol, 1 eq) in ethanol (180 mL) and water (120 mL) was stirred and gave an almost immediate white precipitate. The mixture was heated to reflux to effect solution and then allowed to cool to room temperature slowly. White crystals were collected by filtration and subsequently recrystallised twice from 60% ethanol/40% water to afford the title compound (14.7 g, 49%); ¹H NMR (250 MHz, MeOH-d4) 0.64 (3H, d, 6.7 Hz), 1.07 (3H, d, 6.5 Hz), 1.56 (3H, d, 6.9 Hz), 2.29 (1H, m), 2.97 (1H, d, 10.9 Hz), 4.35 (1H, q, 6.9 Hz), 7.14-7.43 (10H, m).

### Intermediate 14: (S)-3-Methyl-2-phenyl-butyric acid

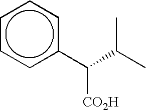

A suspension of intermediate 13 (14.7 g, 49 mmol) in water (100 mL) was acidified with 10% aqueous sulphuric acid and extracted with dichloromethane (3×100 mL). The organic extracts were dried over magnesium sulphate and concentrated in vacuo. Cooling the residue to 0 deg C. afforded the title compound as a white solid (8.65 g, 99%); ¹H NMR (250 MHz, CDCI₃): 0.70 (3H, d, 6.7 Hz), 1.08 (3H, d, 6.5 Hz), 2.33 (1H, m), 3.14 (1H, d, 10.6 Hz), 7.24-7.33 (5H, m); [α]^{23.9}_{D}=+65.2° (c=1 in MeOH).

### Intermediate 15: (S)-3-Methyl-2-phenyl-butyramide

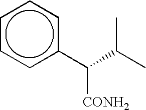

To a solution of intermediate 14 (6.58 g, 37 mmol, 1 eq) in dichloromethane (100 mL) was added oxalyl chloride (4.8 mL, 55 mmol, 1.5 eq) and catalytic dimethylformamide (2 drops). The reaction was stirred at room temperature for 90 minutes and then concentrated in vacuo. The residue was dissolved in THF (60 mL) and cooled to 0 deg C. before aqueous ammonia (32%, 150 mL) was added slowly and the reaction allowed to warm to room temperature. Stirring was continued for 15 hrs, ethyl acetate was then added and stirring continued for an additional 30 minutes. The organic phase was separated, dried over magnesium sulphate and concentrated in vacuo. The residue was dissolved in ethyl acetate, washed with 2N HCl, dried over magnesium sulphate and concentrated in vacuo to afford the title compound as a white solid (6.35 g, 97%); ¹H NMR (250 MHz, DMSO-d6): 0.60 (3H, d, 6.7 Hz), 0.96 (3H, d, 6.5 Hz), 2.24 (1H, m), 2.96 (1H, d, 10.7 Hz), 6.74 (2H, brs), 7.19-7.33 (5H, m); [α]^{29.1}_{D}=+53.0° (c=1 in MeOH).

### Intermediate 16: (S)-2-Methyl-1-phenyl-propylamine

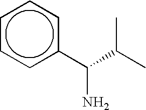

To a solution of bistrifluoroacetoxyiodobenzene (23.1 g, 53 mmol, 1.5 eq) in acetonitrile/water (100 mL, 1:1), was slowly added a solution of intermediate 15 (6.35 g, 36 mmol, 1 eq) in acetonitrile/water (1:1, 100 mL). Stirring was continued for 2 hrs. The acetonitrile was removed in vacuo and the aqueous residue acidified with 30% aqueous H₂SO₄. The reaction mixture was extracted with ether (which was discarded) and then basified with 50% aqueous sodium hydroxide. The basic aqueous phase was extracted with dichloromethane and the resulting organics dried over magnesium sulphate and concentrated in vacuo. The residue was purified by chromatography on silica gel eluting with 5% methanol/ethyl acetate (with 0.5% aq ammonia added) to afford the title compound (3.5 g, 65%); ¹H NMR (250 MHz, CDCI₃): 0.77 (3H, d, 6.8 Hz), 0.98 (3H, d, 6.7 Hz), 1.85 (1H, m), 3.60 (1H, d, 7.2 Hz), 7.20-7.33 (5H, m); [α]^{29.6}_{D}=−10.5° (c=1.25 in DCM).

### Intermediate 17: 1-Cyclobutyl-1-phenyl-methanone oxime

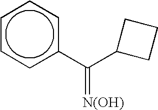

A mixture of 1-cyclobutyl-1-phenyl-methanone (Aldrich Chemical Company) (10.0 g, 62 mmol, 1 eq) and hydroxylamine hydrochloride (6.46 g, 94 mmol, 1.5 eq) in ethanol (60 mL) was treated with a solution of KOH (17.5 g, 312 mmol, 5 eq) in water (30 mL). The mixture was heated at reflux for 24 hrs before cooling and pouring into an ice/water bath. Acidification to pH 1 with conc HCl afforded a white solid which was filtered, washed with water and dried in vacuo. Recrystallisation from ether/petrol gave the title compound as white crystals (9.6 g, 68%); m/z (ES): 176 [M+H]⁺.

Intermediates 18-21 of formula (X) (see Table 1) were prepared in similar manner to intermediate 17.

TABLE 1

| | |
|---|---|
| | (X) |
| 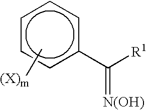 | |

| Intermediate | (X)ₘ | R¹ | m/z |
|---|---|---|---|
| 18 | 2-fluoro | ethyl | APCI 168 |
| | | | [M + H]⁺ |
| 19 | 3-fluoro | ethyl | APCI 168 |
| | | | [M + H]⁺ |
| 20 | 4-fluoro | ethyl | ES 168 |
| | | | [M + H]⁺ |
| 21 | H | cyclopropyl | APCI 162 |
| | | | [M + H]⁺ |

### Intermediate 22: (S)-1-Cyclobutyl-1-phenyl)-methylamine (hydrochloride salt)

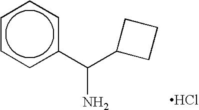

Intermediate 17 (6.18 g, 35 mmol) was dissolved in ethanol (100 mL) and 10% palladium on charcoal catalyst (0.47 g) added. The reaction mixture was hydrogenated at room temperature and atmospheric pressure for 3 hrs. The mixture was filtered and HCl (35 mL of a 1M solution in ether) added. Concentration in vacuo afforded the title compound as a white solid (6.7 g, 96%); ¹H NMR (400 MHz, MeOH-d4) 1.70-2.05 (5H, m), 2.20-2.27 (1H, m), 2.85-2.93 (1H, m), 4.20 (1H, d, J=10 Hz), 7.37-7.46 (5H, m).

Intermediates 23-25 of formula (III) (see Table 2) were prepared in similar manner to intermediate 22.

TABLE 2

| | |
|---|---|
| | (III) |
| 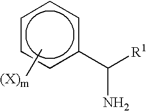 | |

| Intermediate | (X)ₘ | R¹ | ¹H NMR (400MHz) |
|---|---|---|---|
| 23 | 2-fluoro | ethyl | (CDCl₃) 1.10, (3H, t, J=8Hz), 2.53-2.58, |
| | | | (1H, m), 2.77-2.83, (1H, m), 2.98-3.02, |
| | | | (1H, m), 7.06-7.40, (4H, m). |
| 24 | 3-fluoro | ethyl | HCl salt [DMSO] 0.76, (3H, t, J=8Hz), |
| | | | 1.77-1.85, (1H, m), 1.96-2.03, (1H, m), |
| | | | 4.14-4.19, (1H, m), 7.21-7.51, (4H, m), |
| | | | 8.64, (3H, brs). |
| 25 | 4-fluoro | ethyl | (CDCl₃) 0.86, (3H, t, J=7Hz), 1.60-1.71, |
| | | | (2H, m), 3.81, (1H, t, J=7Hz), 6.98- |
| | | | 7.48, (4H, m). |

### Intermediate 26: 1-Cyclopropyl-1-(4-fluoro-phenyl)-methanone oxime

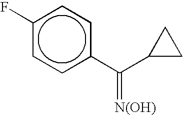

A mixture of cyclopropyl-(4-fluorophenyl) methanone (Aldrich Chemical Company) (6.56 g, 40 mmol, 1 eq), hydroxylamine hydrochloride (4.45 g, 64 mmol, 1.6 eq) and pyridine (30 mL) was stirred at room temperature for 24 hrs. The reaction mixture was concentrated to dryness in vacuo and the residue partitioned between ethyl acetate and water. The organic phase was washed with water and saturated aqueous brine, dried over magnesium sulphate and then concentrated in vacuo. The residue was purified by chromatography on silica gel eluting with ethyl acetate/hexane (1:5 to 1:2 gradient elution) to give the title compound as a colourless oil (6.0 g, 84%). Product a 2:1 mixture of the E and Z geometric isomers; ¹H NMR (400 MHz, DMSO d⁶) 0.49-0.51 (m), 0.68-0.70 (m), 0.74-0.76 (m), 0.86-0.89 (m) (4H in total), 1.68-1.70 (m), 2.11-2.15 (m) (1H in total), 7.11-7.22 (m), 7.40-7.44 (m), 7.53-7.57 (m) (4H in total).

### Intermediate 27: 1-Cyclopropyl-1-(4-fluoro-phenyl)-methanone O-benzyl oxime

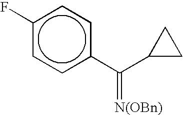

To a solution of intermediate 26 (13.87 g, 77 mmol, 1 eq) in dry DMF (250 mL) at 0 deg C. was added sodium hydride (4.03 g of 60% dispersion in mineral oil, 100 mmol, 1.3 eq) in small portions. When addition was complete the reaction mixture was stirred for an additional 30 mins at 0 deg C. and then treated with a solution of benzyl bromide (17.1 g, 100 mmol, 1.3 eq) in DMF (50 mL) dropwise over a period of 40 mins. The reaction was then allowed to warm to room temperature and stirring continued for 5 hrs. After cooling to 0 deg C., the reaction was quenched with a solution of ethanol/water (10 mL/2 mL). Brine (40 mL) was added and the reaction mixture extracted with diethyl ether (4×75 mL). The organic phases were combined and washed with water and brine and then dried over sodium sulphate. Evaporation to dryness gave a oil which was purified by chromatography on silica gel, eluting with dichloromethane/petroleum ether (40-60 deg C.) (15-50% gradient) to give the title compound as a clear oil (1.89 g, 9%); ¹H NMR (400 MHz, CDCI₃) 0.59-0.62, (2H, m), 0.89-0.94, (2H, m), 2.21-2.28, (1H, m), 5.20, (2H, s), 6.99-7.03, (2H, t), 7.30-7.43, (7H, m)

### Intermediate 28: 1-Cyclopropyl-1-phenyl-methanone O-benzyl oxime

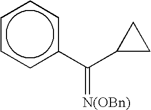

The title compound was prepared in a similar manner to intermediate 27 using intermediate 21 as starting material; m/z (APCI) : 252 [M+H]⁺.

### Intermediate 29: (S)-1-Cyclopropyl-1-(4-fluoro-phenyl)-methylamine

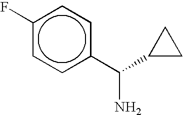

A stirred solution of (S)-2-amino-3-methyl-1,1-diphenol butan-1-ol (1.66 g, 6.5 mmol, 2.5 eq) in anhydrous THF (20 mL) was treated with borane-THF complex (13.2 mL of a 1M solution, 13.2 mmol, 5 eq) over a period of 20 mins. The resulting mixture was stirred at room temperature for 2 hrs, then cooled to 0 deg C. and a solution of intermediate 27 (0.7 g, 2.6 mmol, 1 eq) in THF (20 mL) was added dropwise, keeping the temperature below 0 deg C. When addition was complete, the mixture was stirred at 0 deg C. for 30 mins and then allowed to warm to room temperature and stirred for 18 hrs. The mixture was cooled to 0 deg C. and treated with 5N HCl (10 ml) and stirred at room temperature for 6 hrs. Upon recooling to 0 deg C., the mixture was basified to pH 12 with 2M NaOH solution and then extracted with ethyl acetate. The organic phase was washed with water and brine, dried over sodium sulphate and then concentrated to dryness. The residue was purified by chromatography on silica gel, eluting with 0-5% methanol/dichloromethane to afford the title compound as a colourless oil (0.33 g, 77%); ¹H NMR (400 MHz, CDCI₃) 0.24-0.33, (2H, m), 0.47-0.50, (1H, m), 0.58-0.61, (1H, m),1.05-1.12, (1H, m) 3.18, (1H, d, J=13 Hz), 6.96-7.05, (2H, m), 7.35-7.41, (2H, m).

### Intermediate 30: 1-Cyclopropyl-1-phenyl-methylamine

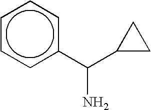

The title compound was prepared in a similar manner to intermediate 29 using intermediate 28 as starting material and borane THF complex as reducing agent; HCl salt: ¹H NMR (400 MHz, MeOH-d4) 0.41-0.44 (1H, m), 0.58-0.67 (2H, m), 0.79-0.85 (1H, m), 1.35-1.45 (1H, m), 3.58 (1H, d, J=10 Hz), 7.41-7.49 (5H, m).

### Intermediate 31: 1-Cyclopropyl-1-(2-fluoro-phenyl)-methanol

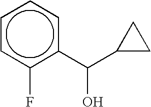

2-Fluorobenzaldehyde (1 g, 8.1 mmol, 1 eq) was dissolved in ether (5 mL) and cooled in an ice bath to 0 deg C. Cyclopropylmagnesium bromide (17 mL of a 0.5M solution in THF, 8.5 mmol, 1.05 eq) was added and the reaction mixture allowed to warm to room temperature and stirred for 15 hrs. Saturated ammonium chloride solution was slowly added and additional water added to dissolve all inorganic residues. The aqueous phase was extracted with ether (×3) and the combined organics washed with water and brine, dried over magnesium sulphate and then concentrated in vacuo. The residue was purified by chromatography on silica gel eluting with 0-30% ether/petrol to afford the title compound (0.79 g, 59%); ¹H NMR (400 MHz, CDCI₃) 0.38-0.52 (3H, m), 0.60-0.66 (1H, m), 1.25-1.29 (1H, m), 2.07 (1H, dd, J=4 Hz, 1 Hz), 4.37 (1H, dd, J=8 Hz, 4 Hz), 7.00-7.05 (1H, m), 7.12-7.17 (1H, m), 7.21-7.28 (1H, m), 7.51-7.55 (1H, m).

### Intermediate 32: 1-Cyclopropyl-1-(3-fluoro-phenyl)-methanol

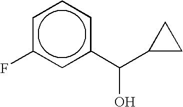

The title compound was prepared in a similar manner to intermediate 31 using 3-fluorobenzaldehyde; m/z (APCI): 149 [MH⁺-H₂O]⁺.

### Intermediate 33: 1-(1-Azido-1-cyclopropyl-methyl)-2-fluoro-benzene

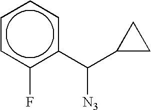

Intermediate 31 (0.79 g, 4.7 mmol, 1 eq) was dissolved in THF (10 mL) and cooled in an ice/water bath. Diphenylphosphoryl azide (1.43 g, 5.2 mmol, 1.1 eq) and DBU (0.79 g, 5.2 mmol, 1.1 eq) were added dropwise and the reaction mixture allowed to warm to room temperature. Stirring was continued for 15 hrs. The reaction was partitioned between 5M HCl, water and ether. The organic phase was separated and washed with water, saturated aqueous sodium bicarbonate solution and brine, dried over magnesium sulphate and concentrated in vacuo. The residue was purified by chromatography on silica gel eluting with 0-30% ether/petrol to afford the title compound; m/z (APCI): 164 [MH⁺-N₂]⁺.

### Intermediate 34: 1(1-Azido-1-cyclopropyl-methyl)-3-fluoro-benzene

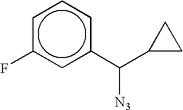

The title compound was prepared in a similar manner to intermediate 33 using intermediate 32; m/z (APCI): 164 [MH⁺-N₂]⁺.

### Intermediate 35: 1-Cyclopropyl-1-(2-fluoro-phenyl)-methylamine (hydrochloride salt)

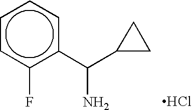

Intermediate 33 (0.303 g, 1.6 mmol, 1 eq) was dissolved in THF (5 mL) and triphenylphosphine (497 mg, 1.9 mmol, 1.2 eq) was added and stirring continued at room temperature for 18 hrs. Water (1 mL) and THF (2 mL) were added and the mixture heated to reflux for 8 hrs and then allowed to cool to room temperature. The reaction mixture was partitioned between ether and 2M HCl. The organic phase was washed with water and discarded. The combined aqueous phases were cooled in an ice water bath and then basified with sodium hydroxide and extracted with ether (×2). The combined organic phases were washed with brine and dried over magnesium sulphate and concentrated in vacuo. Addition of HCl (1M in ether) afforded the title compound as a white solid (132 mg, 41%); ¹H NMR (400 MHz, MeOH-d4) 0.38-0.43 (1H, m), 0.60-0.68 (2H, m), 0.83-0.86 (1H, m), 1.42-1.51 (1H, m), 3.83 (1H, d, J=10 Hz), 7.20-7.25 (1H, m), 7.28-7.32 (1H, m), 7.46-7.50 (1H, m), 7.54-7.59 (1H, m).

### Intermediate 36: 1-Cyclopropyl-1-(3-fluoro-phenyl)-methylamine (hydrochloride salt)

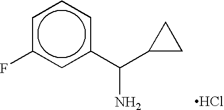

The title compound was prepared in a similar manner to intermediate 35 using intermediate 34; ¹H NMR (400 MHz, MeOH-d4) 0.43-0.47 (1H, m), 0.59-0.70 (2H, m), 0.79-0.88 (1H, m), 1.32-1.40 (1H, m), 3.62 (1H, d, J=10 Hz), 7.14-7.19 (1H, m), 7.25-7.32 (2H, m), 7.46-7.50 (1H, m).

### Intermediate 37: 3-Methyl-2-(3-fluoro-phenyl)-quinoline carboxylic acid

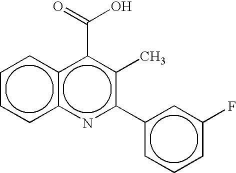

Isatin (9.7 g, 66 mmol, 1 eq) was stirred in glacial acetic acid (180 mL) at room temperature and 3-fluoropropiophenone (10 g, 66 mmol, 1 eq) added. The reaction was then heated to 75 deg C. After 10 minutes, conc HCl (66 mL) was added (to give a dark red solution) and the reaction subsequently heated at 105 deg C. for 15 hrs. After cooling to room temperature, water (330 mL) was added to afford a beige solid which was collected by filtration and washed with water. Further solid precipitated from the mother liquors on standing. After 2 hrs a second batch of material was collected and washed with water and ether. Combining both batches afforded 4.87 g (26%) of the title compound; m/z (APCI) : 282 [M+H]⁺.

### Intermediate 38: 3-methyl-2-phenyl-quinoline carboxylic acid

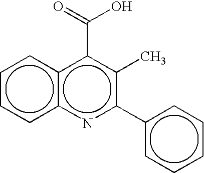

The title compound was prepared in a similar manner to intermediate 37 using propiophenone as starting material; ¹H NMR [400 MHz, DMSO-d6] 2.38, (3H,s), 7.48-8.06, (9H, m).

### Intermediate 39: 3-Methyl-2-(3-fluoro-phenyl)quinoline carboxylic acid, methyl ester.

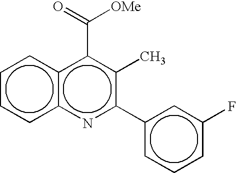

To a solution of intermediate 37 (35 g, 125 mmol, 1 eq) in dichloromethane (350 mL) was added DMF (2-3 drops) followed by the slow addition of oxalyl chloride (37 mL, 423 mmol, 3.4 eq). The reaction was stirred at room temperature for 15 hrs and then concentrated to dryness in vacuo. The residue was dissolved in MeOH/CH₂Cl₂ and stirred for 90 minutes and then concentrated in vacuo. The residue was partitioned between dichloromethane and 10% aqueous sodium bicarbonate, the organic phase separated, dried over magnesium sulphate and concentrated in vacuo. Chromatography of the crude product on silica, eluting with 25% petroleum ether/dichloromethane—100% dichloromethane afforded the title compound (24.4 g, 66%) as a pale yellow solid; m/z (APCI): 296.2 [M+H]⁺.

### Intermediate 40: 3-Methyl-2-phenyl-quinoline carboxylic acid, methyl ester.

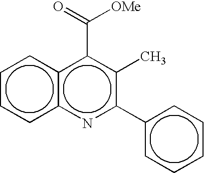

The title compound was prepared in a similar manner to intermediate 39 using intermediate 38 as starting material to afford the title compound; ¹H NMR [400 MHz, CDCI₃] 2.40 (3H, s), 4.08 (3H, s), 7.44-7.73 (8H, m), 8.14 (1H, d).

### Intermediate 41: 3-Bromomethyl-2-(3-fluoro-phenyl)-quinoline-4-carboxylic acid methyl ester.

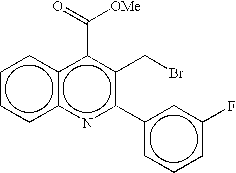

To a solution of intermediate 39 (14.4 g, 50 mmol) in carbon tetrachloride (300 mL) was added N-bromo succinimide (10.5 g, 60 mmol, 1.2 eq). The reaction was heated to reflux before AIBN (500 mg) was added in one portion. Heating was continued for 15 hrs and then cooled to room temperature and concentrated in vacuo. The residue was partitioned between ethyl acetate and water. The organic phase was repeatedly washed with water (to remove last traces of succinimide), dried over magnesium sulphate and concentrated in vacuo. Trituration with ether afforded the title compound as a pale yellow solid (18 g, 97%); m/z (APCI): 374/376 [M+H]⁺.

### Intermediate 42: 3-Bromomethyl-2-phenyl-quinoline4-carboxylic acid methyl ester.

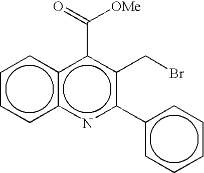

The title compound was prepared in a similar manner to intermediate 41 using intermediate 40 as starting material and using acetonitrile as the solvent to afford the title compound; m/z (ES+): 356, 358 [M+H]⁺.

### Intermediate 43: 2-(3-Fluoro-phenyl)-3-[1,2,3]triazol-2-ylmethyl-quinoline-4-carboxylic acid methyl ester and intermediate 44: 2-(3-Fluoro-phenyl)-3-[1,2,3]triazol-1-ylmethyl-quinoline-4-carboxylic acid methyl ester.

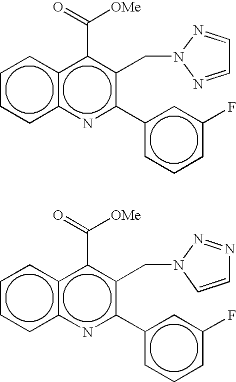

To a cooled solution of 1,2,3 triazole (0.628 g, 9.11 mmol, 1.3 eq) in DMF (15 mL) was added sodium hydride (0.308 g of a 60% dispersion in oil, 7.71 mmol, 1.1 eq) over a period of 2 hours, keeping the temperature below 10 deg C. When addition was complete, the reaction was allowed to warm to room temperature and stirred for 90 minutes. This solution was added dropwise over 90 minutes to a solution of intermediate 41 (2.62 g, 7.01 mmol, 1 eq) in DMF. After 1 hr the reaction mixture was concentrated in vacuo and the residue partitioned between ethyl acetate and 10% aqueous sodium bicarbonate solution. The organic phase was washed with 10% aqueous sodium bicarbonate, dried over magnesium sulphate and concentrated in vacuo. The residue was purified on silica gel eluting with 0-25% ethyl acetate/dichloromethane to give intermediate 43 as a yellow solid (0.774 g, 31%) m/z (ES+): 363 [M+H]⁺ and intermediate 44 (0.877 g, 35%); m/z (ES+) : 363 [M+H]⁺.

Intermediates 45-56 of formula (IVa) (see Table 3) were prepared in a similar manner to intermediates 43 and 44 using intermediate 41 or 42 as starting material and an appropriate nitrogen containing heterocycle.

TABLE 3

| | |
|---|---|
| | (IVa) |
| 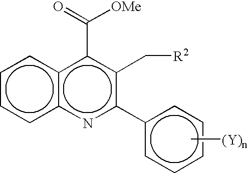 | |

| Intermediate | R² | (Y)ₙ | m/z |
|---|---|---|---|
| 45 | 1,2,3 triazol-2-yl | H | 345 [M + H]⁺ |
| 46 | 1,2,3 triazol-1-yl | H | 345 [M + H]⁺ |
| 47 | 1,2,4 triazol-1-yl | H | 345 [M + H]⁺ |
| 48 | tetrazol-2-yl | H | 346 [M + H]⁺ |
| 49 | tetrazol-1-yl | H | 346 [M + H]⁺ |
| 50 | tetrazol-2-yl | 3-F | 364 [M + H]⁺ |
| 51 | tetrazol-1-yl | 3-F | 364 [M + H]⁺ |
| 52 | 5-methyl tetrazol-2-yl | H | 360 [M + H]⁺ |
| 53 | 5-methyl tetrazol-1-yl | H | 360 [M + H]⁺ |
| 54 | pyrazol-1-yl | H | 344 [M + H]⁺ |
| 55 | 3-trifluoromethyl pyrazol-1-yl | H | 412 [M + H]⁺ |
| 56 | 3,5 -dimethyl pyrazol-1-yl | H | 372 [M + H]⁺ |

### Intermediate 57: 2-(3-Fluoro-phenyl)-3-[1,2,3]triazol-2-ylmethyl-quinoline-4-carboxylic acid.

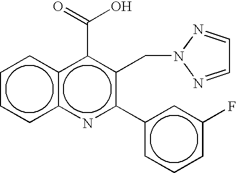

To a solution of intermediate 43 (8.0 g, 22 mmol, 1 eq) in ethanol (100 mL) was added lithium hydroxide monohydrate (2.8 g, 66 mmol, 3 eq) in water (100 mL) portionwise. The reaction mixture was heated to reflux for 4 hrs, cooled to room temperature and then concentrated in vacuo. The residue was acidified with 2N HCl and subsequently filtered and dried in vacuo to afford the title compound (5.7 g, 75%); m/z (APCI) 349 [M+H]⁺.

Intermediates 58-70 of formula (V) (see Table 4) were prepared in a similar manner to intermediate 57.

TABLE 4

| | |
|---|---|
| | (V) |
| 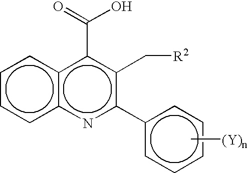 | |

| Intermediate | R² | (Y)ₙ | m/z |
|---|---|---|---|
| 58 | 1,2,3 triazol-1-yl | 3-fluoro | 349 [M + H]⁺ |
| 59 | 1,2,3 triazol-2-yl | H | 331 [M + H]⁺ |
| 60 | 1,2,3 triazol-1-yl | H | 331 [M + H]⁺ |
| 61 | 1,2,4 triazol-1-yl | H | 331 [M + H]⁺ |
| 62 | tetrazol-2-yl | H | 332 [M + H]⁺ |
| 63 | tetrazol-1-yl | H | 332 [M + H]⁺ |
| 64 | tetrazol-2-yl | 3-fluoro | 350 [M + H]⁺ |
| 65 | tetrazol-1-yl | 3-fluoro | 350 [M + H]⁺ |
| 66 | 5-methyl tetrazol-2-yl | H | 344 [M + H]⁺ |
| 67 | 5-methyl tetrazol-1-yl | H | 344 [M + H]⁺ |
| 68 | pyrazol-1-yl | H | 330 [M + H]⁺ |
| 69 | 3-trifluoromethyl pyrazol-1-yl | H | 398 [M + H]⁺ |
| 70 | 3,5 -dimethyl pyrazol-1-yl | H | 358 [M + H]⁺ |

### Intermediate 71: (S)-1-Phenyl propyl amine

Intermediate 71 was purchased from Lancaster Chemical Company.

### EXAMPLES

### Example 1

### 2-(3-Fluoro-phenyl)-3-[1,2,3]triazol-2-ylmethyl-quinoline4-carboxylic acid ((S)-1-cyclopropyl-1-phenyl-methyl)-amide.

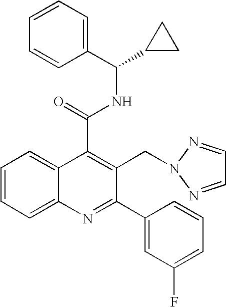

A solution of intermediate 57 (5.5 g, 16 mmol, 1 eq), intermediate 10 (3.2 g, 17 mmol, 1.05 eq) and DIPEA (8.3 mL) in DMF were stirred for 20 minutes before cooling in an ice/water bath. HATU (6.1 g, 16 mmol, 1 eq) was added portionwise and the reaction allowed to slowly warm to room temperature. Stirring continued for a further 48 hrs before concentrating in vacuo. The residue was partitioned between ethyl acetate and 10% aqueous sodium carbonate. The organic phase was washed with 10% aqueous sodium bicarbonate solution, dried over magnesium sulphate and then concentrated in vacuo. The residue was purified by chromatography (Jones flashmaster) eluting with 20-50% ethyl acetate/petroleum ether to give the title compound (4.95 g, 60%) as an off white solid; ¹H NMR [250 MHz, DMSO-d6, 353K] [HCl salt] 0.33-0.61 (4H, m), 1.20-1.54 (1H, m), 4.56 (1H, t, J=8.5), 5.66 (2H, brs), 7.03-7.42 (9H, m), 7.45 (2H, s), 7.58-7.71 (1H, m), 7.80-7.86 (2H, m), 8.05 (1H, dd, J=9.13 and 1.2), 9.06 (1H, d, J=15.0).

Examples 2-43 of general formula (Ib) (see Table 5) were prepared in a similar manner to example 1 from the starting material indicated.

| | |
|---|---|
| | (Ib) |
| 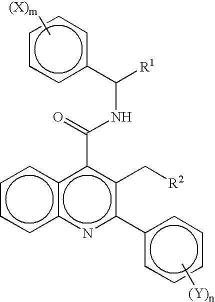 | |

| Ex | R¹ | R² | (X)ₘ | (Y)ₙ | starting material | m/z |
|---|---|---|---|---|---|---|
| 2 | cyclopropyl | 1,2,3-triazol-1- | H | 3-fluoro | intermediates | 496 |
| | | yl | | | 30 and 58 | [M + H]⁺ |
| 3 | (S)- | 3-CF₃-1- | H | H | intermediates | 527 |
| | cyclopropyl | pyrazolyl | | | 10 and 69 | [M + H]⁺ |
| 4 | ethyl | 1,2,3-triazol-1- | 4-fluoro | 3-fluoro | intermediates | 484 |
| | | yl | | | 25 and 58 | [M + H]⁺ |
| 5 | (S)-ethyl | 3,5-dimethyl- | H | H | intermediate 70 | 475 |
| | | pyrazol-1-yl | | | and 71 | [M + H]⁺ |
| 6 | cyclopropyl | 1,2,3-triazol-2- | H | H | intermediates | 460 |
| | | yl | | | 30 and 59 | [M + H]⁺ |
| 7 | cyclopropyl | 1,2,3-triazol-2- | 2-fluoro | H | intermediates | 478 |
| | | yl | | | 35 and 59 | [M + H]⁺ |
| 8 | cyclopropyl | 1,2,3-triazol-1- | H | H | intermediates | 460 |
| | | yl | | | 30 and 60 | [M + H]⁺ |
| 9 | ethyl | 1,2,3-triazol-2- | 3-fluoro | 3-fluoro | intermediates | 484 |
| | | yl | | | 24 and 57 | [M + H]⁺ |
| 10 | ethyl | 1,2,3-triazol-2- | 3-fluoro | H | intermediates | 466 |
| | | yl | | | 24 and 59 | [M + H]⁺ |
| 11 | ethyl | 1,2,3-triazol-1- | 2-fluoro | H | intermediates | 466 |
| | | yl | | | 23 and 60 | [M + H]⁺ |
| 12 | cyclopropyl | 1,2,3-triazol-1- | 2-fluoro | H | intermediates | 478 |
| | | yl | | | 35 and 60 | [M + H]⁺ |
| 13 | (S)- | 1,2,3-triazol-2- | 3-fluoro | 3-fluoro | intermediates | 496 |
| | cyclopropyl | yl | | | 11 and 57 | [M + H]⁺ |
| 14 | (S)-ethyl | 1,2,3-triazol-2- | H | 3-fluoro | intermediates | 466 |
| | | yl | | | 57 and 71 | [M + H]⁺ |
| 15 | (S)-ethyl | 5-Me-tetrazol- | H | H | intermediates | 463 |
| | | 1-yl | | | 67 and 71 | [M + H]⁺ |
| 16 | (S)-ethyl | 1-pyrazolyl | H | H | intermediates | 447 |
| | | | | | 68 and 71 | [M + H]⁺ |
| 17 | cyclopropyl | 1,2,3-triazol-2- | 3-fluoro | H | intermediates | 478 |
| | | yl | | | 36 and 59 | [M + H]⁺ |
| 18 | (S)- | 1,2,3-triazol-1- | H | H | intermediates | 462 |
| | isopropyl | yl | | | 16 and 60 | [M + H]⁺ |
| 19 | ethyl | 1,2,3-triazol-1- | 3-fluoro | 3-fluoro | intermediates | 484 |
| | | yl | | | 24 and 58 | [M + H]⁺ |
| 20 | (S)-ethyl | tetrazol-2-yl | H | H | intermediates | 449 |
| | | | | | 62 and 71 | [M + H]⁺ |
| 21 | cyclopropyl | 1,2,3-triazol-1- | 3-fluoro | H | intermediates | 478 |
| | | yl | | | 36 and 60 | [M + H]⁺ |
| 22 | (S)-ethyl | 1,2,3-triazol-1- | H | 3-fluoro | intermediates | 466 |
| | | yl | | | 58 and 71 | [M + H]⁺ |
| 23 | (S)- | 1,2,3-triazol-2- | H | H | intermediates | 462 |
| | isopropyl | yl | | | 16 and 59 | [M + H]⁺ |
| 24 | ethyl | 1,2,3-triazol-1- | 3-fluoro | H | intermediates | 466 |
| | | yl | | | 24 and 60 | [M + H]⁺ |
| 25 | (S)- | 5-Me-tetrazol- | 3-fluoro | H | intermediates | 492 |
| | cyclopropyl | 2-yl | | | 11 and 66 | [M + H]⁺ |
| 26 | (S)- | [1,2,3]-triazol- | 4-fluoro | H | intermediates | 478 |
| | cyclopropyl | 2-yl | | | 29 and 59 | [M + H]⁺ |
| 27 | (S)-ethyl | [1,2,3]-triazol- | H | H | intermediates | 448 |
| | | 1-yl | | | 60 and 71 | [M + H]⁺ |
| 28 | cyclobutyl | [1,2,3]-triazol- | H | 3-fluoro | intermediates | 492 |
| | | 2-yl | | | 22 and 57 | [M + H]⁺ |
| 29 | (S)- | [1,2,3]-triazol- | 4-fluoro | H | intermediates | 478 |
| | cyclopropyl | 1-yl | | | 29 and 60 | [M + H]⁺ |
| 30 | (S)- | [1,2,3]-triazol- | H | 3-fluoro | intermediates | 478 |
| | cyclopropyl | 1-yl | | | 10 and 58 | [M + H]⁺ |
| 31 | (S)- | tetrazol-2-yl | H | H | intermediates | 461 |
| | cyclopropyl | | | | 10 and 62 | [M + H]⁺ |
| 32 | (S)-ethyl | [1,2,3]-triazol- | H | H | intermediates | 448 |
| | | 2-yl | | | 59 and 71 | [M + H]⁺ |
| 33 | (S)- | [1,2,3]-triazol- | H | H | intermediates | 460 |
| | cyclopropyl | 2-yl | | | 10 and 59 | [M + H]⁺ |
| 34 | cyclobutyl | [1,2,3]-triazol- | H | 3-fluoro | intermediates | 492 |
| | | 1-yl | | | 22 and 58 | [M + H]⁺ |
| 35 | (S)- | tetrazol-2-yl | 3-fluoro | H | intermediates | 479 |
| | cyclopropyl | | | | 11 and 62 | [M + H]⁺ |
| 36 | (S)- | pyrazol-1-yl | 3-fluoro | H | intermediates | 477 |
| | cyclopropyl | | | | 11 and 68 | [M + H]⁺ |
| 37 | (S)- | [1,2,3]-triazol- | 3-fluoro | H | intermediates | 478 |
| | cyclopropyl | 2-yl | | | 11 and 59 | [M + H]⁺ |
| 38 | cyclobutyl | [1,2,3]-triazol- | H | H | intermediates | 474 |
| | | 1-yl | | | 22 and 60 | [M + H]⁺ |
| 39 | (S)-ethyl | tetrazol-1-yl | H | H | intermediates | 449 |
| | | | | | 63 and 71 | [M + H]⁺ |
| 40 | (S)-ethyl | [1,2,4]-triazol- | H | H | intermediates | 448 |
| | | 1-yl | | | 61 and 71 | [M + H]⁺ |
| 41 | cyclobutyl | [1,2,3]-triazol- | H | H | intermediates | 474 |
| | | 2-yl | | | 22 and 59 | [M + H]⁺ |
| 42 | (S)-ethyl | 5-Me-tetrazol- | H | H | intermediates | 463 |
| | | 2-yl | | | 66 and 71 | [M + H]⁺ |
| 43 | ethyl | [1,2,3]-triazol- | 4-fluoro | H | intermediates | 466 |
| | | 1-yl | | | 25 and 60 | [M + H]⁺ |
| 44 | cyclopropyl | [1,2,3]-triazol- | H | 3-fluoro | intermediates | 478 |
| | | 1-yl | | | 30 and 58 | [M + H]⁺ |
| 45 | (S)- | tetrazol-2-yl | H | 3-fluoro | intermediates | 479 |
| | cyclopropyl | | | | 10 and 64 | [M + H]⁺ |

Biological Assays

Measurement of NK Binding Affinity

The NK binding affinity of the compounds of the invention was determined using the following scintillation proximity assay (SPA) (see H. M. Sarau et al, J. Pharmacol. Experimental Therapeutics 1997, 281(3), 1303-1311; H. M. Sarau et al, J. Pharmacol. Experimental Therapeutics 2000, 295(1), 373-381; G. A. M. Giardina et al J.Med.Chem 1999, 42, 1053-1065). ¹²⁵I Substance P, ¹²⁵I NKA and ¹²⁵I [MePhe7]-NKB were used in the binding SPA of NK₁, NK₂ and NK₃ receptor, respectively. Polystrene Leadseeker WGA-SPA beads (Amersham Biosciences) were mixed with plasma membrane prepared from CHO cell lines expressing NK1, NK₂ or NK₃ in a bead/membrane ratio of 20:1 (w/w) in assay buffer (75 mM Tris pH 7.8, 75 mM NaCl, 4 mM MnCl₂, 1 mM EDTA, 0.05% Chaps, 1 mM PMSF). The mixture was placed on ice for 30 minutes to allow the formation of membrane/bead complex before BSA was added to a final concentration of 1%. After another 30 minutes incubation on ice, the bead/membrane complex was washed twice and suspended in assay buffer. ¹²⁵I-labelled ligands were then added to the bead/membrane complex. 30 μl of the resulting mixture was then dispensed into each well of a Nalgen NUNC 384-well plate with 1 μl compound pre-dispensed in 50% DMSO. The plates were then sealed and pulse spun at 1100 rpm. After 3 hours incubation at room temperature with shaking, the plates were spun for 2 min at 1100 rpm and measured in Viewlux imager (PerkinElmer) for 5 minutes with a 618-nm filter. Inhibition of radioactive ligand binding to its respective receptor was measured by the reduction of signal. pKᵢ was calculated using K_{d} of each radioactive ligand determined in a separate experiment.

Measurement of Brain Exposure

Compounds of the invention were orally dosed (3 mg/kg) to rats as 1% methylcellulose (w/v) suspensions. The rats were sacrificed after set time intervals and the concentration of the compound of the invention in brain homogenate was determined by protein precipitation followed by LC-MS-MS analysis of the extracts against standards prepared in brain homogenate. A graph of brain concentration against time was plotted over a 12 hr period. The area under the curve (AUC, units=hours.ng/g brain) was taken as a measure of brain exposure.

The therapeutic potential of the compounds of the invention can be assessed by measurement of the reversal of NK₃ agonist driven behaviours (e.g. contralateral turning in gerbils as described in Life Sciences 1995, 56, PL27-PL32 and Can. J. Physiol. Pharmacol. 2002, 80, 482-488; or guinea pig wet dog shakes as described in Br. J. Pharmacol. 1997, 122, 715-725) or by mechanistic correlates (e.g. electrophysiology of the dopamine cell firing as described in Gueudet et al., Synapse, 1999, 33, 71-79).

The compounds of the invention are potent NK₃ receptor antagonists. The compounds of the invention bind selectively to the NK₃ receptor in preference to the NK₁ and NK₂ receptors. As discussed hereinabove, the compounds of the invention have greater in vivo brain exposure.

The examples described herein gave a pKi for NK₃ of greater than 7.5.

| | | | | Brain Exposure |
|---|---|---|---|---|
| | NK₃ | NK₂ | NK₁ | (AUC, h · ng/g |
| Example | pKi | pKi | pKi | brain) |
| 1 | 8.0 | 6.5 | 6.0 | 1896 |
| 35 | 8.4 | 6.6 | 5.9 | 2361 |
| 37 | 8.6 | 6.8 | 6.2 | 2091 |
| 45 | 8.0 | 6.6 | 5.9 | 1900 |

1. A compound of formula (I), a pharmaceutically acceptable salt, solvate or prodrug thereof 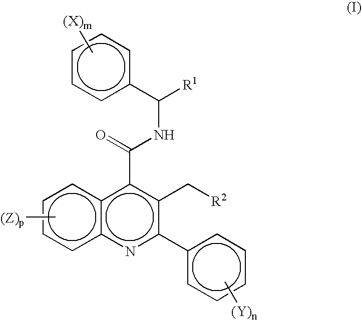
wherein
R¹ is C₁₋₆alkyl, C₃₋₆cycloalkyl or acetyl;
R² is N-linked pyrazolyl, triazolyl or tetrazolyl each of which may be substituted by C₁₋₄alkyl or perfluoro C₁₋₄alkyl;
m, n and p, which may be the same or different, are 0, 1 or 2; and
X, Y and Z are fluoro.

2. A compound according to claim 1 wherein R¹ is cyclopropyl.

3. A compound according to claim 1 wherein R² is N-linked triazolyl or N-linked tetrazolyl.

4. A compound according to claim 1 wherein p is 0.

5. A compound according to claim 1 wherein m and n, which may be the same or different, are 0 or 1.

6. A compound according to claim 1 wherein either a) m is 0 and n is 1, or b) m is 1 and n is 0.

7. A compound according to claim 1 wherein when m and/or n are 1, X and/or Y are attached to the meta-position of the phenyl groups.

8. A compound, pharmaceutically acceptable salt, solvate or prodrug thereof according to claim 1 wherein the stereochemistry is shown in formula (Ia):

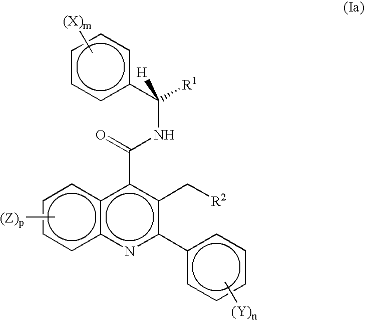

9. A compound according to claim 1, wherein the compound is:
2-(3-Fluoro-phenyl)-3-[1,2,3]triazol-2-ylmethyl-quinoline-4-carboxylic acid ((S)-1-cyclopropyl-1-phenyl-methyl)-amide (Example 1);
2-Phenyl-3-[1,2,3]triazol-2-ylmethyl-quinoline-4-carboxylic acid [(S)-1-cyclopropyl-1-(3-fluoro-phenyl)-methyl]-amide (Example 17);
2-Phenyl-3-tetrazol-2-ylmethyl-quinoline-4-carboxylic acid [(S)-1-cyclopropyl-1-(3-fluoro-phenyl)-methyl]-amide (Example 35); and
2-(3-Fluoro-phenyl)-3-tetrazol-2-ylmethyl-quinoline-4-carboxylic acid ((S)-1-cyclopropyl-1-phenyl-methyl)-amide (Example 45).
